(19) United States
(12) Patent Application Publication
(10) Pub. No.: US 2016/0100767 A1
(43) Pub. Date: Apr. 14, 2016

(54) HEARTBEAT AND PULSE MEASURING SYSTEM AND METHOD THEREOF

(71) Applicant: Cloud Care Technologies Co. Ltd., Taipei City (TW)

(72) Inventors: Bo-Jau KUO, Taipei (TW); Cheng-Han WU, Taipei (TW); Wei-Yi LI, Taipei (TW); Vincent I-Te HSIEH, Taipei (TW)

(21) Appl. No.: 14/588,220

(22) Filed: Dec. 31, 2014

(30) Foreign Application Priority Data
Oct. 9, 2014 (TW) 103135212

(51) Int. Cl. A61B 5/024; A61B 5/00

(52) U.S. Cl. CPC A61B 5/02433; A61B 5/7225; A61B 5/0004

(57) ABSTRACT

The present invention provides a heartbeat and pulse measuring system comprising a detecting unit, a processing unit and an outputting unit. The detecting unit includes a first light-emitting element, a second light-emitting element and a detecting module. The first light-emitting element emits a first frequency beam to a user's body to generate a first scatter light signal. The second light-emitting element emits a second frequency beam to the body to generate a second scatter light signal. The detecting module receives first scattered light signal and the second scattered light signal to covert into a first current signal and a second current signal respectively. The processing unit analyzes and calculates the intensity difference associated with time between the first current signal and the second current signal to produce an analysis information, then the analysis information is outputted by the outputting unit.

### CROSS-REFERENCE TO RELATED APPLICATIONS

This Non-provisional application claims priority under 35 U.S.C. §119(a) on Patent Application No(s). 103135212 filed in Taiwan, Republic of China, Oct. 9, 2014, the entire contents of which are hereby incorporated by reference.

### FIELD OF THE INVENTION

At least one embodiment in accordance with the present invention relates to the measuring systems and methods for operating thereof. More particularly, at least one embodiment relates to the heartbeat and pulse measuring systems and methods for operating thereof.

### BACKGROUND OF THE INVENTION

Detections of heartbeat and pulse are conventionally performed with multiple electrode pads simultaneously attached on the skin of a subject. Those electrode pads constitute part of a stationary device and are hard to be incorporated into wearable devices. Recently, several wearable devices were proposed with photoplenthysmographs which, alternately, determine heartbeat and pulse by measuring the change in light absorption. The photoplethysmography technology, however, shows low resilience to the dynamical activities of a subject. Various noises are constantly produced with the motions of a subject and therefore decline the accuracy of the pulse measurement.

Wearable devices are featured in its high mobility, and the photoplethysmography technology used in wearable devices should be stable under different situations. Conventional photoplenthysmographs are nevertheless vulnerable to the activities of a subject. External factors such as the level of ambient light and the variation of temperature and internal factors such as the contraction of muscle tissue also largely affect the accuracy of the pulse measurement.

Accordingly, there is a need for heartbeat and pulse measuring systems and methods having improvements in the aforementioned defects.

### SUMMARY OF THE INVENTION

Some embodiments of the present invention provide a heartbeat and pulse measuring system comprising a detecting unit, a processing unit, and an output unit.

The detecting unit in accordance with some embodiments of the present invention comprises a first light-emitting element, a second light-emitting element, and a detecting module. As attaching with a subject, the first light-emitting element emits a first frequency beam toward the subject to generate a first scattered light signal and the second light-emitting element emits a second frequency beam toward the subjected to generate a second scattered light signal. In particular, the first scatter light signal and the second scatter light signal each represents a variation of light intensity over time.

The detecting module in accordance with some embodiments of the present invention is configured for receiving the first scattered light signal and the second scattered light signal from the subject. The detecting module further converts the first scattered light signal and the second scattered light signal into a first current signal and a second current signal respectively.

In some embodiments, the first light-emitting element, the second light-emitting element, and the detecting module are spaced at, but not limited to, equal intervals.

The processing unit in accordance with some embodiments of the present invention is connected with the detecting unit. The processing unit comprises an amplifier, a filter, a processing module, and a first transmission module. The amplifier is configured for amplifying the first current signal and the second current signal. The filter is configured for filtering noises off the first current signal and the second current signal. The processing module is configured for generating an analysis information by processing the first current signal and the second current signal. The first transmission module is configured for transmitting the analysis information to the output unit. In particular, the analysis information herein is obtained by computing the intensity difference between the first current signal and the second current signal in time domain.

The output unit in accordance with some embodiments of the present invention is coupled with the processing unit. The output unit comprises a second transmission module which is configured for receiving the analysis information. The output unit may further convert the analysis information into a heartbeat and pulse information.

Some embodiments of the present invention provide a method for operating the heartbeat and pulse measuring systems. The method comprises several steps. In the first step, a first light-emitting element and a second light-emitting element emit a first frequency beam and a second frequency beam to a subject respectively to obtain a first scattered light signal and a second scattered light signal. The subject is attaching with both the first light-emitting element and the second light-emitting element, and the first scattered light signal and the second scattered light signal each represents a variation of light intensity in time domain. The second step is associated with analog-to-digital conversion; the first scattered light signal and the second scattered light signal received from the subject are converted into a first current signal and a second current signal respectively. The first current signal and the second current signal are further amplified and/or filtered in the third step. The last two steps are obtaining an analysis information by computing the intensity difference between the first current signal and the second current signal in time domain and transmitting the analysis information to an output unit for a conversion from the analysis information to a heartbeat and pulse information, respectively.

Some embodiments in accordance with the present invention provide accurate heartbeat and pulse by recording two scattered light signals which are in different wavelengths. The two scattered light signals are used to attenuate the common mode noises induced by external factors and internal factors. The detecting unit in some embodiments may be configured in a portable device. Even with a subject under vigorous motions, the detecting unit is capable of monitoring the heartbeat and pulse of the subject.

The embodied heartbeat and pulse measuring systems and embodied methods provide improvements over the prior arts.

### BRIEF DESCRIPTION OF THE DRAWINGS

FIG. 1 is a block diagram illustrating a heart rate measuring system, according to some embodiments of the present invention.

FIG. 2 is a schematic diagram illustrating a detecting unit, according to some embodiments of the present invention.

FIG. 3 is a schematic diagram illustrating a computing process based on a first current signal and a second current signal, according to some embodiments of the present invention.

FIG. 4 is a schematic photoplethysmogram representing an analysis information, according to some embodiments of the present invention.

FIG. 5 is a flow diagram illustrating a method for operating heart rate measuring devices, according to some embodiments of the present invention.

### DETAILED DESCRIPTION OF THE INVENTION

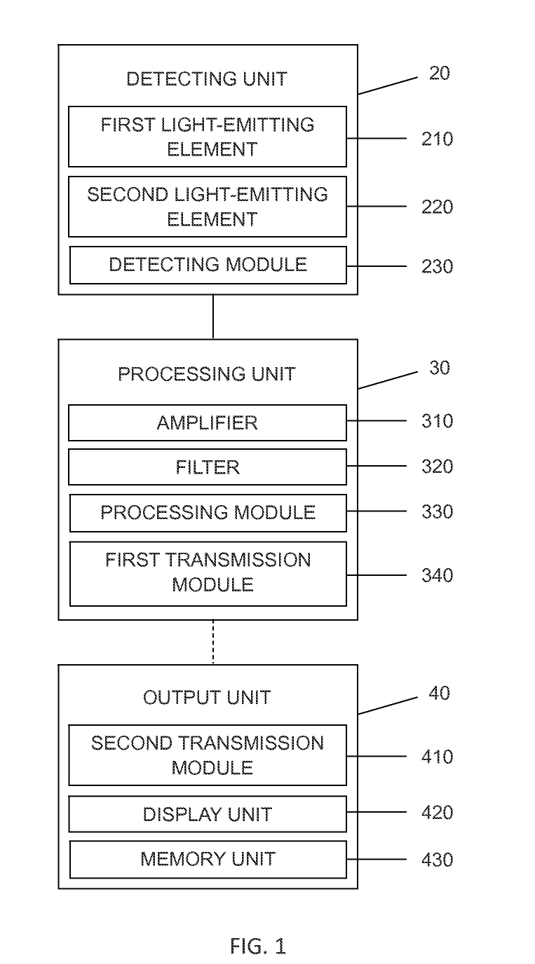

FIG. 1 is a block diagram illustrating a heartbeat and pulse measuring system, according to some embodiments of the present invention. The heartbeat and pulse measuring system comprises a detecting unit 20, a processing unit 30, and an output unit 40.

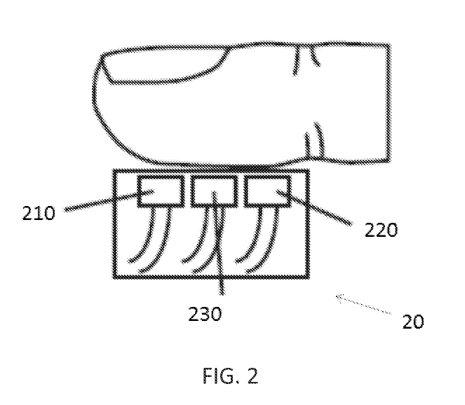

FIG. 2 is schematic diagram illustrating a detecting unit, according to some embodiments of the present invention. The detecting unit 20 comprises a first light-emitting element 210, a second light-emitting element 220, and a detecting module 230. As attaching with a subject, the first light-emitting element emits a first frequency beam toward the subject to generate a first scattered light signal and the second light-emitting element emits a second frequency beam toward the subjected to generate a second scattered light signal. In particular, the first scatter light signal and the second scatter light signal each represents a variation in light intensity over time.

In some embodiments, the first light-emitting element 210 is, but not limited to, an infrared light-emitting diode (LED) and the first frequency beam is, but not limited to, infrared light. The second light-emitting element 220 is an LED with any wavelength. Preferably, the second light-emitting element 220 is, but not limited to, a far infrared LED or a green LED, and the second frequency beam is, but not limited to, far infrared light or green light respectively.

In some embodiments, the detecting module 230 is, but not limited to, disposed between the first light-emitting element 210 and the second light-emitting element 220. The detecting module 230 is configured for receiving the first scattered light signal and the second scattered light signal from the subject, and converting the first scattered light signal and the second scattered light signal into a first current signal and a second current signal respectively.

In some embodiments, the first light-emitting element 210, the second light-emitting element 220, and the detecting module 230 are spaced at, but not limited to, equal intervals.

The processing unit 30 in accordance with some embodiments of the present invention is connected with the detecting unit 20. The processing unit comprises an amplifier 310, a filter 320, a processing module 330, and a first transmission module 340. The amplifier 310 is configured for amplifying the first current signal and the second current signal. The filter 320 is configured for filtering noises off the first current signal and the second current signal. The processing module 330 is configured for generating an analysis information by processing the first current signal and the second current signal. The first transmission module 340 is configured for transmitting the analysis information to the output unit 40. In particular, the analysis information herein is obtained by computing the intensity difference between the first current signal and the second current signal in time domain.

In some embodiments, the amplifier 310 is configured for, but not limited to, amplifying strength of the first current signal and the second current signal. The filter 320 is configured for, but not limited to, filtering noises from the first current signal and the second current signal.

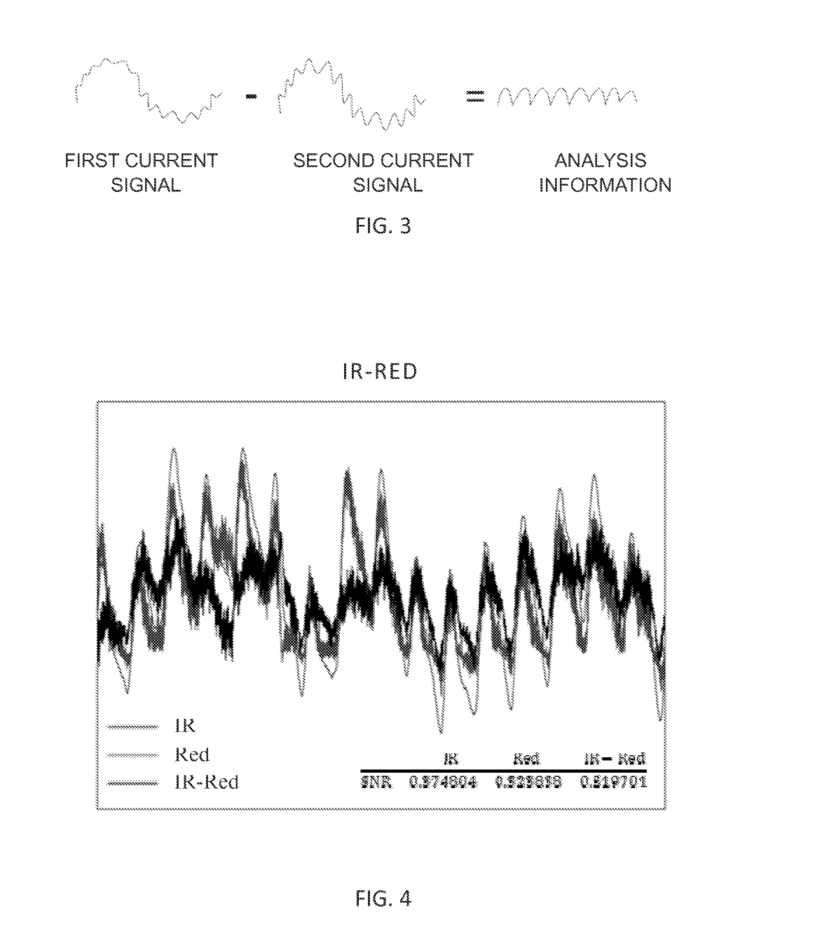

FIG. 3 is a schematic diagram illustrating a computing process based on a first current signal and a second current signal, according to some embodiments of the present invention. The computing process is, but not limited to, for attenuating the common mode noises. The processing module 330 may compute the intensity difference between the first current signal and the second current signal in time domain and obtain an analysis information.

FIG. 4 is a schematic photoplethysmogram representing an analysis information, according to some embodiments of the present invention. In these embodiments, the first current signal obtained from a light beam from a far infrared LED is colored in blue, and the second current signal obtained from a light beam from an infrared LED is colored in red. The signal-to-noise ratios of the first current signal and the second current signal are 0.374804 and 0.323838 respectively. A data illustrated in black, representing an analysis information computed from the intensity difference between the first current signal and the second current signal, shows a signal-to-noise ratio which is 0.519701. Indicated by FIG. 4, the baseline wander noise is significantly suppressed and the signal-to-noise ratio is largely improved in the analysis information obtained by the systems and methods in accordance with the present invention.

The output unit 40 in accordance with some embodiments of the present invention is coupled with the processing unit 30. The output unit 40 comprises a second transmission module 410 which is configured for receiving the analysis information. The output unit 40 may convert the analysis information into a heartbeat and pulse information.

In some embodiments, the output unit 40 comprises a display unit 420 and a memory unit 430. The display unit 420 and the memory unit are configured for, but not limited to, displaying and storing the analysis information respectively.

In some embodiments, the detecting unit 20, the processing unit 30 and the output unit 40 of a heartbeat and pulse measuring system are not co-existed in a same device. For example, a small optical detector comprises a detecting unit 20 and a processing unit 30 may obtain an analysis information by attaching to the fingertip of a subject. The analysis information may be transmitted to a portable device, a smart phone, via wireless transceivers. In the smart phone, the analysis information could be displayed on the screen, saved to the memory, or performed with other actions.

In some embodiments, the detecting unit 20, the processing unit 30 and the output unit 40 in heartbeat and pulse measuring system are co-existed in a same device. For example, a smart phone. The smart phone comprises a detecting unit 20, a processing unit 30, a screen, and even a memory. One may tap a fingertip to the detecting unit 20 to initiate the processes. Analyzing of the first current signal and the second current signal are performed by the processing unit 30 of the same smart phone. Even the analysis information can be displayed by the screen and saved to the memory of the smart phone.

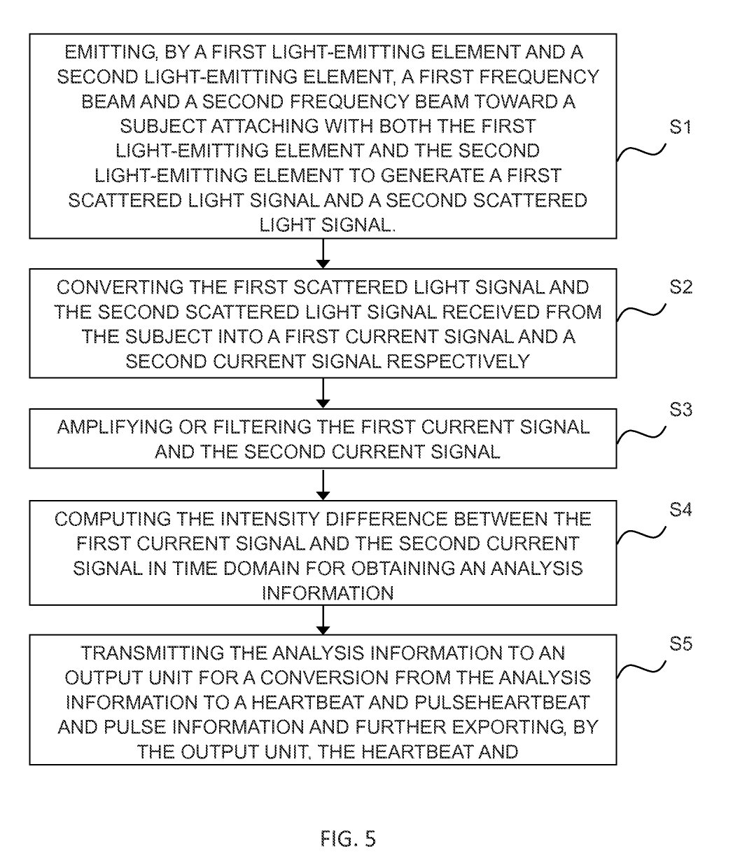

FIG. 5 is a flow diagram illustrating a method for operating heart rate measuring devices, according to some embodiments of the present invention. The method for operating heart rate measuring devices comprises several steps. In the first step (S1), a first light-emitting element and a second light-emitting element emit a first frequency beam and a second frequency beam to a subject respectively to obtain a first scattered light signal and a second scattered light signal. The subject is attaching with the first light-emitting element and the second light-emitting element both, and the first scattered light signal and the second scattered light signal each represents a variation in light intensity in time domain. The first scattered light signal and the second scattered light signal received from the subject are converted into a first current signal and a second current signal respectively in the second step (S2); and the first current signal and the second current signal is further amplified and/or filtered in the third step (S3). The last two steps are the step 4 (S4) of obtaining an analysis information by computing the intensity difference between the first current signal and the second current signal in time domain and the step 5 (S5) of transmitting the analysis information to an output unit for a conversion from the analysis information to a heartbeat and pulse information, respectively.

In some embodiments, the analysis information is transmitted to the output unit through, but not limited to, a wired network or a wireless network. In some embodiments, the analysis information is exported, by the output unit, to, but not limited to, a display unit for displaying or a memory unit for storing.

Some embodiments in accordance with the present invention provide accurate heartbeat and pulse by recording two scattered light signals which are in different wavelengths. The two scattered light signals are used to attenuate the common mode noises induced by external factors and internal factors. The detecting unit in some embodiments may be configured in a portable device. Even with a subject under vigorous motions, the detecting unit is capable of monitoring the heartbeat and pulse of the subject.

There are many inventions described and illustrated herein. The present inventions are neither limited to any single aspect nor embodiment thereof, nor to any combinations and/or permutations of such aspects and/or embodiments. Moreover, each of the aspects of the present inventions, and/or embodiments thereof, may be employed alone or in combination with one or more of the other aspects of the present inventions and/or embodiments thereof. For the sake of brevity, many of those permutations and combinations will not be discussed separately herein.

What the claims are:

1. A heartbeat and pulse measuring system, comprising:
a detecting unit, comprising:
a first light-emitting element, attaching with a subject, for emitting a first frequency beam toward the subject to generate a first scattered light signal, wherein the first scatter light signal is a variation of light intensity in time domain;
a second light-emitting element, attaching with the subject, for emitting a second frequency beam toward the subjected to generate a second scattered light signal, wherein the second scatter light signal is the variation in light intensity in time domain; and
a detecting module, for receiving the first scattered light signal and the second scattered light signal and converting the first scattered light signal and the second scattered light signal into a first current signal and a second current signal;
a processing unit, connected with the detecting unit, comprising:
a processing module, for obtaining an analysis information by computing the intensity difference between the first current signal and the second current signal in time domain; and
a first transmission module, for transmitting the analysis information; and
an output unit, coupled with the processing unit, for receiving the analysis information for a conversion from the analysis information to a heartbeat and pulse heartbeat and pulse information and exporting the heartbeat and pulse information.

2. The heartbeat and pulse measuring system as claimed in claim 1, wherein the first light-emitting element, the second light-emitting element, and the detecting module are spaced at equal intervals.

3. The heartbeat and pulse measuring system as claimed in claim 1, wherein the first frequency beam is infrared light.

4. The heartbeat and pulse measuring system as claimed in claim 3, wherein the second frequency beam is one selected from the group consisting of far infrared light or green light.

5. The heartbeat and pulse measuring system as claimed in claim 1, wherein the processing unit further comprises an amplifier configured for amplifying the first current signal and the second current signal.

6. The heartbeat and pulse measuring system as claimed in claim 1, wherein the processing unit further comprises a filter configured for filtering noises off the first current signal and the second current signal.

7. The heartbeat and pulse measuring system as claimed in claim 1, wherein the output unit further comprises a second transmission module configured for receiving the analysis information from the first transmission module.

8. A method for measuring heartbeat and pulse systems, comprising:
emitting, by a first light-emitting element and a second light-emitting element, a first frequency beam and a second frequency beam toward a subject attaching with both the first light-emitting element and the second light-emitting element to generate a first scattered light signal and a second scattered light signal, wherein the first scattered light signal and the second scattered light signal each is a variation of light intensity in time domain;
converting the first scattered light signal and the second scattered light signal received from the subject into a first current signal and a second current signal respectively;
computing the intensity difference between the first current signal and the second current signal in time domain for obtaining an analysis information; and
transmitting the analysis information to an output unit for a conversion from the analysis information to a heartbeat and pulse information and further exporting, by the output unit, the heartbeat and pulse information.

9. The method for operating heartbeat and pulse systems as claimed in claim 8, wherein the first current signal and the second current signal were pretreated with amplification or filtration before the step of computing.

10. The method for operating heartbeat and pulse systems as claimed in claim 8, wherein the analysis information is transmitted via a wireless network in the step of transmitting.
